(19) United States
(12) Patent Application Publication
(10) Pub. No.: US 2025/0312963 A1
(43) Pub. Date: Oct. 9, 2025

(54) Plant and Method for Producing and Treating Containers

(71) Applicant: Krones AG, Neutraubling (DE)

(72) Inventors: Ralf URBAN, Teugn (DE); Jochen HIRDINA, Regensburg (DE)

(21) Appl. No.: 19/097,752

(22) Filed: Apr. 1, 2025

(30) Foreign Application Priority Data
Apr. 3, 2024 (DE) 10 2024 109 306.4

(51) Int. Cl. B29C 49/46; A61L 2/20; A61L 2/24; B29C 49/42; B29C 49/78; B29K 67/00; B29L 31/00

(52) U.S. Cl. CPC B29C 49/46; A61L 2/202; A61L 2/208; A61L 2/24; B29C 49/42418; B29C 49/42822; B29C 49/78; B29C 2049/4697; B29C 2049/7861; B29K 2067/003; B29L 2031/7158

(57) ABSTRACT

A plant for producing and treating containers includes a clean room, a production machine for producing preforms, a blow-molding machine arranged downstream of the production machine for shaping the preforms into containers and a container treatment machine arranged downstream of the blow-molding machine for treating the containers. The production machine, the blow-molding machine and the container treatment machine are arranged in the clean room, and each component of the production machine, the blow-molding machine and the container treatment machine that comes into physical contact with a preform or a container consists of or comprises sterilization-proof material.

### CROSS-REFERENCE TO RELATED APPLICATIONS

This application claims priority to, and the benefit of, German Patent Application No. 10 2024 109 306.4, filed Apr. 3, 2024, the contents of which are incorporated by reference in their entirety.

### TECHNICAL FIELD

The present invention relates to a plant for producing and treating containers.

### BACKGROUND ART

Plants for producing and/or treating containers are sufficiently well known in the prior art.

For example, DE 10 2011 056 293 A1 discloses a plant comprising an injection-molding machine and downstream devices, in particular a forming device and a labeling machine, which are arranged in a clean room and thus ensure that the preforms produced in the injection-molding machine are sterile.

EP 4 122 872 A1 further discloses a plant in a protective housing so that certain clean room requirements can be ensured.

DE 10 2015 105 994 A1 describes an interlocked plant in which an injection-molding machine, a blow-molding machine and a filling device are arranged within a clean room, thus allowing the most sterile conditions possible to be realized.

Although known plants in principle allow the production and further treatment of containers under clean room conditions, the introduction of non-sterile components or materials during subsequent processes can lead to contamination of the containers, which can have a negative impact on the quality of the containers and in particular the products.

### BRIEF SUMMARY

Aspects of the present invention relate to a plant for producing and treating containers as well as a method for producing and treating containers by a plant with which the subsequent introduction of contaminants can also be treated with the lowest possible amount of maintenance effort.

The plant for producing and treating containers according to one aspect of the invention comprises a clean room, a production machine for producing preforms, a blow-molding machine arranged downstream of the production machine for shaping the preforms into containers and a container treatment machine arranged downstream of the blow-molding machine for treating the containers, wherein the production machine, the blow-molding machine and the container treatment machine are arranged in the clean room and wherein each component of the production machine, the blow-molding machine and the container treatment machine that comes into physical contact with a preform or a container consists of or comprises sterilization-proof material.

In this context, containers are to be understood to mean in particular bottles or cans or tubes or similar objects that can be produced from preforms.

The components of the production machine, the blow-molding machine and the container treatment machine that come into physical contact with a preform or a container are to be understood as the components or parts of these machines that come into direct physical contact with the preforms or containers, i.e. that directly touch them on their inner or outer surface without any other materials in between. The sterilization-proof material is to be understood in particular as a material which does not corrode or experience any other deterioration in quality or material degeneration when these components are exposed to a sterilization medium.

With this plant, in addition to the sterility of the atmosphere in the clean room that is fundamentally guaranteed, it is also possible to carry out additional sterilization steps if, for example, the atmosphere and/or the components are accidentally or intentionally contaminated, which could have a detrimental effect on the quality of the containers produced. The need to replace components as a result of wear is avoided by using sterilization-proof material.

In one embodiment it is provided that the sterilization-proof material is resistant to exposure to a gaseous and/or liquid sterilization medium. The gaseous or liquid sterilization medium preferably has antibacterial and/or antiviral and/or fungicidal properties. This allows for reliable sterilization of the components and at the same time prevents wear and tear thereof, even with frequent sterilization.

In particular, it can be provided that the sterilization medium is H₂O₂ and/or O₃ and/or peracetic acid. These sterilization media are usually used in the beverage processing industry or in the food, cosmetics and medical engineering industries in general. Materials that are resistant thereto can therefore be used particularly advantageously.

It can be provided that the plant comprises a clean room sterilization device which can apply a sterilization medium to at least one region of the clean room. The clean room sterilization device can in particular be configured in such a way that it applies a sterilization medium (for example H₂O₂ or ozone or peracetic acid) to the entire clean room in order to restore the clean room quality, for example after plant changeover operations. The application of sterilization medium can preferably take place during a cleaning phase or a cleaning operation, wherein the cleaning operation is an operating mode of the plant during which no containers are produced and/or treated. This advantageously prevents produced and/or treated containers from being influenced by the sterilization medium.

It can be provided that the plant comprises a preform sterilization device which can apply a sterilization medium to preforms.

If there is a risk of contamination of the preforms or insufficient sterilization of the preform material when producing the preforms (for example due to the temperature at which the preforms are produced being too low), the preform sterilization device can advantageously be used to ensure that the preforms are sterile before they are shaped into containers, wherein wear is advantageously minimized by using sterilization-proof materials for the components (here in particular grippers or clamps for conveying the preforms).

In particular, it can be provided that the plant comprises a control unit which is configured to control the preform sterilization device on the basis of a parameter relating to the application of the sterilization medium to the preforms, which parameter is indicative of the sterility of the preforms.

The control unit can be configured as a computer with an associated memory and/or processor, which, for example by comparing a measured indicative parameter of one or more preforms with a limit value, in which the measured indicative parameter exceeding or falling below this limit value indicates a risk of contamination, controls the preform sterilization device in order to reduce the risk of contamination by dispensing sterilization medium. This can preferably be configured in such a way that the preform sterilization device is used to apply the sterilization medium to the preforms only if there is a risk of contamination, i.e. the indicative parameter is indicative of such a contamination risk (i.e. accordingly exceeds or falls below the limit value).

It can be provided that the parameter indicative of the sterility of the preforms comprises a temperature at which the preforms are produced and/or a temperature at which the preforms are discharged from the production machine. In the production machine, the temperature used to produce the preforms usually has a sterilizing effect. However, this is guaranteed only when a certain temperature is exceeded. If the production process is carried out at too low a temperature or if the preforms leave the production machine at too low a temperature, this may indicate that not all germs that may be present on the preform material have been killed. The production temperature or the discharging temperature can therefore be used as reliable parameters indicative of the sterility of the preform to control sterilization processes.

In one embodiment, it is provided that the plant comprises a heating device for controlling the temperature of the preforms and wherein the heating device comprises at least one microwave radiator. Microwave radiators are usually made of materials that are resistant to common sterilization media such as H₂O₂ or O₃ and can therefore advantageously be used to heat the preforms before feeding them to the blow-molding machine or to apply a desired temperature profile thereto, while at the same time minimizing maintenance effort and/or wear. Alternatively or additionally, the heating device can also control the temperature of the preforms by one or more laser sources and/or by exposing the preforms to infrared radiation (for example by one or more infrared radiators).

The container treatment machine may comprise a filler for filling containers with a product and/or a sealer for sealing full containers and/or a labeling machine for attaching a label to a container and/or a direct printing machine for applying a printed image to a container. In particular, filling and/or sealing the containers should be carried out preferably under sterile conditions. If the components in question are made of or comprise sterilization-proof material, the container treatment machine can advantageously also be sterilized without causing undesirable wear or maintenance effort.

The plant may comprise a preform cooling device for cooling preforms with a sterile first cooling medium and/or a container cooling device for cooling containers with a sterile second cooling medium. This preform cooling device (which can use sterile air or nitrogen as a cooling medium, for example) avoids the introduction of further contamination risks.

It can be provided that the component coming into physical contact with a preform or a container comprises at least one of a gripper, a clamp, a blow mold, a conveying rail, a filling element, a centering device, a base plate or rotary plate, a sealing element, an air conveyor arranged in the clean room for transporting containers and/or preforms.

These components are used typically to convey or treat containers. If these or at least the surfaces thereof that come into contact with the container or preform are provided with a sterilization-proof material, maintenance effort and wear are reduced even if sterilization within the clean room is necessary.

According to another aspect of the invention, a method for producing and treating containers by a plant is further provided, the plant comprising a clean room, a production machine for producing preforms, a blow-molding machine arranged downstream of the production machine for shaping the preforms into containers and a container treatment machine arranged downstream of the blow-molding machine for treating the containers, wherein the production machine, the blow-molding machine and the container treatment machine are arranged in the clean room and wherein each component of the production machine, the blow-molding machine and the container treatment machine that comes into physical contact with a preform or a container consists of or comprises sterilization-proof material, the method involving producing the preforms by the production machine, feeding the preforms to the blow-molding machine and shaping the preforms into containers and feeding the containers to the container treatment machine and treating the containers, wherein the preforms and containers come into physical contact with the components.

This process ensures reliable operation of the plant for producing and treating containers, particularly under high cleanliness conditions.

It can be provided that the plant comprises a clean room sterilization device which can apply a sterilization medium to at least one region of the clean room, and wherein the method comprises applying the sterilization medium to the region; and/or

that the plant comprises a preform sterilization device which can apply a sterilization medium to preforms, and wherein the method involves applying the sterilization medium to at least one preform.

These devices can be used to provide additional sterilization if necessary, for example if the clean room has been opened for maintenance purposes or if the preforms do not meet the necessary sterility requirements after being produced.

In particular, in the second alternative of this embodiment, it can be provided that the plant comprises a control unit which is configured to control the preform sterilization device on the basis of a parameter relating to the application of the sterilization medium to the preforms, which parameter is indicative that the preforms are sterile, wherein the method involves controlling the preform sterilization device for applying sterilization medium to the preforms by the control unit if the indicative parameter exceeds or falls below a limit value.

The limit value may, for example, be indicative of the fact that the preforms are sterile, with this value being exceeded or fallen below (in particular only one of the two) may then represent a risk of contamination of the preforms. Selectively exposing the preforms only in cases where a contamination risk is established on the basis of the indicative parameter saves sterilization medium and reduces the load on the plant.

It can be provided that the plant is a plant according to any one of the preceding embodiments. This realizes the advantages described above of the plant with respect to the production and treatment of containers.

### BRIEF DESCRIPTION OF THE FIGURES

FIG. 1 shows a schematic view of a plant for producing and treating containers; and

FIG. 2a-2g show example embodiments of components comprising sterilization-proof material.

### DETAILED DESCRIPTION

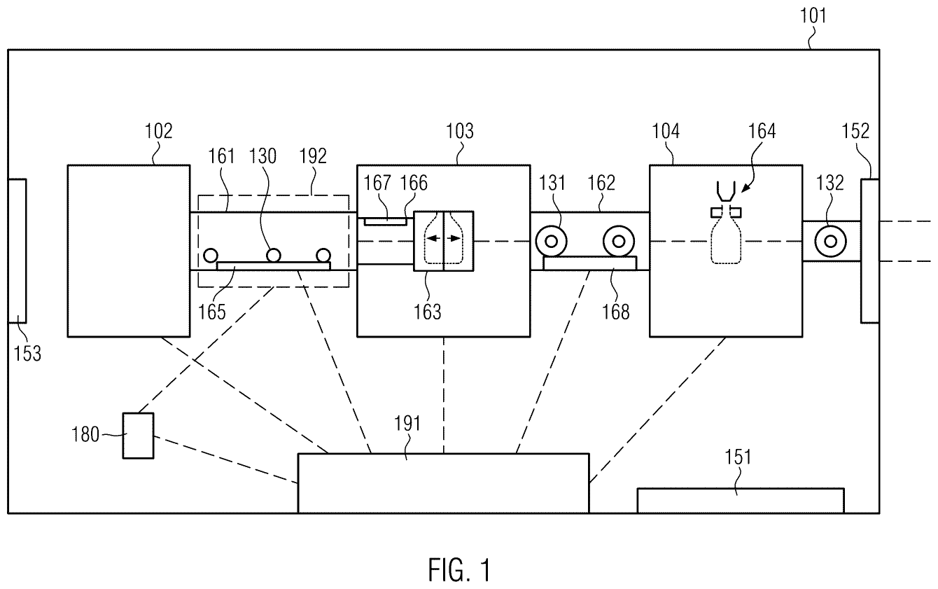

FIG. 1 shows an embodiment of a plant 100 for producing and treating containers 131. The plant 100 comprises a clean room 101. This clean room 101 can be configured in such a way that it can realize or fulfill clean room requirements necessary for the production and treatment of containers in accordance with the known clean room classes. For this purpose, common measures such as suitable air supply and/or removal as well as filter systems can be provided. These are known in principle from the prior art.

The plant 100 further comprises a production machine 102 arranged in the clean room 101, with which preforms 130 can be produced. The production machine is not limited in terms of its design, but can preferably be configured as an injection-molding machine, compression machine or compression-molding machine. These designs are known in principle from the prior art. In general, it is provided that the preforms 130 are produced in the production machine 102 from a preform starting material (for example PET) by a tool, wherein the preform starting material is heated to such an extent that it usually liquefies and then this liquefied preform material is introduced into suitable molds of the tool in order to produce preforms from the liquefied material by cooling.

Furthermore, the plant comprises a blow-molding machine 103 in the clean room, which can produce a container 131, such as a bottle, from a preform 130 by a blow mold 163. Blow-molding machines are known in principle and the preferred type is a stretch blow-molding machine. This can, but does not have to, be configured as a rotary machine, on the periphery of which a plurality of blow molds are arranged so as to rotate about an axis of rotation, each of which can receive preforms and form them into a container while applying blowing pressure and optionally by stretching with a stretching rod.

Downstream of the blow-molding machine 103, another container treatment machine 104 is arranged in the clean room 101, which can treat the containers 131 produced. Preferably, the container treatment machine 104 comprises a filler for filling the containers 131 with a product (such as a beverage, a cosmetic product or a medicinal product). This is shown schematically here by a filling element 164 that pours a product into the container. The container treatment machine 104 may preferably comprise not only a filler, but additionally or alternatively also a sealer (not shown here) which seals the full container 131. This makes it possible to realize a plant 100 for producing, filling and sealing the containers in the clean room 101 such that all method steps up to sealing the container, after which the risk of contamination of the product no longer exists, are realized under clean room conditions.

In order to transport the preforms 130 to the blow-molding machine, a transport device 161 can be provided which can receive the preforms from the production machine 102 and feed them to the blow-molding machine 103. The transport device 161 can be, for example, a linear conveyor, in particular an air conveyor with associated air conveying rails, wherein the preforms can be received between the rails, for example lying on their support ring, and transported in the direction of the blow-molding machine by the action of air. Alternatively, one or more star wheels can also be provided here, which can pick up the preforms with clamps or grippers and transport them from the production machine to the blow-molding machine.

Analogously, a corresponding transport device 162 can be connected to the blow-molding machine 103, which feeds the containers 131 to the downstream container treatment machine 104. The container treatment machine 104 can also be connected to a transport device which can, for example, remove the treated containers 132 from the clean room via a removal opening 152 and can, for example, feed them to further treatment steps, such as a labeling machine or a direct printing machine or a container decorating machine in which containers are printed on or labeled in some other way, e.g. using a laser labeling unit. The removal opening 152 can be configured as a lock subjected to overpressure so that passing the treated containers 132 through the removal opening 152 results in no or only very little contamination of the interior of the clean room 101.

Analogously, a feed device 153 can be provided, which can also be configured as a lock and can feed preform starting material from outside the clean room 101 to the production machine or to a storage container for preform material that is assigned to the production machine.

Furthermore, an entrance 151 can be provided through which operating personnel can enter the interior of the clean room 101, for example to carry out maintenance work.

Optionally, a preform cooling device 165 can be arranged, for example, in the region of the transport device 161, with which the preforms can be exposed to a cooling medium, preferably a sterile one (for example sterile air or nitrogen). Analogously, a container cooling device 168 can alternatively or additionally be provided, for example in the region of the transport device 162, by which the containers 131 produced in the blow-molding machine 103 can be cooled before they are fed to the downstream container treatment machine. This can also preferably apply a sterile (second) cooling medium (for example sterile air or nitrogen), which can be the same cooling medium which the preform cooling device supplies to the preforms, if provided, to the containers 131.

While the provision of the clean room 101 and the machines of the plant located therein make it possible to produce and treat the containers in a generally sterile manner, unintentional or unavoidable contamination can also occur. This is the case, for example, when operators have to enter the clean room to carry out maintenance work (e.g. replacing defective parts or changing the product type by replacing individual components). In addition, there may continue to be a risk of contamination during the production of the preforms from preform material that cannot usually be fed to the plant in a sterile state if the temperatures used in the production machine when producing the preforms are below a temperature at which sterilization can be reliably achieved, or if the time for producing the preforms is too short to achieve the most complete sterilization possible of the preform material.

This may require further sterilization of the clean room 101 and/or the preforms 130 to ensure that the containers produced and treated meet the sterility requirements.

In order to make this possible and at the same time to minimize the maintenance effort or wear of the plant 100, it is provided that some, preferably all, of the components of the production machine, the blow-molding machine and the container treatment machine that come into physical contact with the preforms or the containers consist of sterilization-proof material or comprise sterilization-proof material. This applies to all components that come into direct contact with the preforms or containers without any material in between, as these may also need to be sterilized if they have come into contact with a potentially contaminated preform or container or if any other contamination has entered the clean room 101.

Preferably, the sterilization-proof material is arranged at least on one surface of the components that comes into contact with the preforms and/or containers, or these components are made entirely from the sterilization-proof material (e.g. stainless steel). Sterilization-proof material is understood here in particular to mean a material that is resistant to the sterilization media usually used in the food industry and/or the cosmetics industry and/or the pharmaceutical industry, i.e. that neither corrodes nor degenerates in any other way, in particular does not chemically react with the relevant sterilization media. This includes, for example, stainless steel, which is resistant to common sterilization media such as H₂O₂ (hydrogen peroxide) and ozone (O₃). The corresponding material may also or alternatively be resistant to peracetic acid. Alternatively or additionally, the sterilization-proof material may be resistant to fungicides.

The components that come into contact with the preforms and/or containers are to be understood as including, by way of example but not exclusively, the transport devices shown in FIG. 1 or their components that come into contact with the containers or preforms, as well as, for example, the blow mold 164 and the filling element 164. If they are made of or at least comprise sterilization-proof materials, subsequent additional sterilization of either these components or the preforms or containers can also be carried out during operation without affecting these components, allowing them to be used without or with greatly reduced wear.

While, in principle, mechanical sterilization of the plant 100 does not necessarily have to be provided, and this can also be carried out manually as required by operators with suitable equipment, for example, the plant can comprise a clean room sterilization device 191 which is configured to apply a sterilization medium to at least one region of the clean room (for example specifically the production machine or the blow-molding machine or the container treatment machine). However, it is preferable for the clean room sterilization device to be able to apply a sterilization medium to the entire clean room 101 or its interior with. Preferably, this is a gaseous sterilization medium such as ozone or hydrogen peroxide.

The clean room sterilization device 191 can be used, for example, to sterilize inside the clean room 101 again completely or re-establish clean room quality after maintenance work has been carried out, during which, for example, operators have entered the clean room through the entrance 151 and thus contaminated the air within the clean room. It can be provided that a control unit 180 (for example the central control unit of the plant 100 or a separate control unit) controls the clean room sterilization device 191 on the basis of a signal indicating that the entrance 151 is closed again in order to apply sterilization medium thereby into the at least the region of the clean room 101. For example, the control unit can implement a sterilization cycle by the clean room sterilization device 191, which sterilizes the interior of the clean room 101 in which the production machine 102, the blow-molding machine 103 and the container treatment machine 104 are arranged, for example by introducing sterilization medium in a certain concentration inside the clean room for several hours. (One or more) suitable sensors (not shown here) can measure the concentration of the sterilization medium and transfer it to the control unit via suitable connections for data exchange so that the control unit can either increase or reduce the amount of sterilization medium introduced by the clean room sterilization device 191 per unit of time in order to realize the desired concentration of the sterilization medium in the clean room.

Alternatively or additionally, a preform sterilization device 192 may also be provided, which may be arranged such that it can apply a sterilization medium to the preforms. For example, the preform sterilization device 192 can be integrated into the production machine 102 and/or arranged in the region of the transport device 161 so that it can apply sterilization medium (liquid or gaseous) to the preforms. This can be used particularly advantageously to additionally sterilize the preforms and the components coming into contact therewith if, for example, during the production of the preforms a parameter indicative that the preforms are sterile indicates that the preforms and thus also the components coming into contact with these preforms are at risk of being contaminated.

A suitable indicative parameter for this purpose is, for example, a temperature at which the preforms are produced within the production machine 102 and/or a discharging temperature of the preforms at which the preforms leave the production machine 102 (for example, the temperature they have when they are fed to the transport device 161). (One or more) suitable sensors (not shown here) can determine this temperature and pass it on to the control unit via suitable connections for data exchange and/or the control unit can derive the production temperature and/or the discharging temperature from the operating parameters with which the preforms are produced in the production machine 102.

In principle, the control unit 180 can establish whether there is a risk of contamination by comparing the indicative parameter with a limit value. If this limit value is exceeded or fallen below (depending on whether the limit value being exceeded or undershoot is indicative of an existing risk of contamination of the preforms), the control unit can selectively control the preform sterilization device 192 such that it applies a sterilization medium to the preforms. Preferably, the indicative parameter can be dynamically monitored so that the sterilization medium is only applied to the preforms and preferably to the components in contact therewith if a risk of contamination is detected; otherwise, no sterilization medium is applied. This reduces the consumption of sterilization medium and at the same time the load on the plant.

Optionally, a heating device 166 for controlling the temperature of the preforms can also be provided, which can be arranged, for example (as shown here) as part of the blow-molding machine 103 or upstream of the blow-molding machine in order to control the temperature of the preforms, in particular to provide them with a temperature profile, before they are shaped into containers.

Preferably, the heating device 166 comprises one or more microwave radiators 167, which can control the temperature of the preforms by applying microwaves. Microwave radiators are usually made of materials that are resistant to common sterilization media so that the wear and tear on the plant caused by additional exposure to sterilization media is thereby also reduced. Alternatively or additionally, embodiments of the heating device having one or more laser sources for controlling the temperature of the preforms and/or having one or more infrared radiators for controlling the temperature of the preforms with infrared radiation are also possible. Any combinations of the aforementioned embodiments of the heating device are also conceivable.

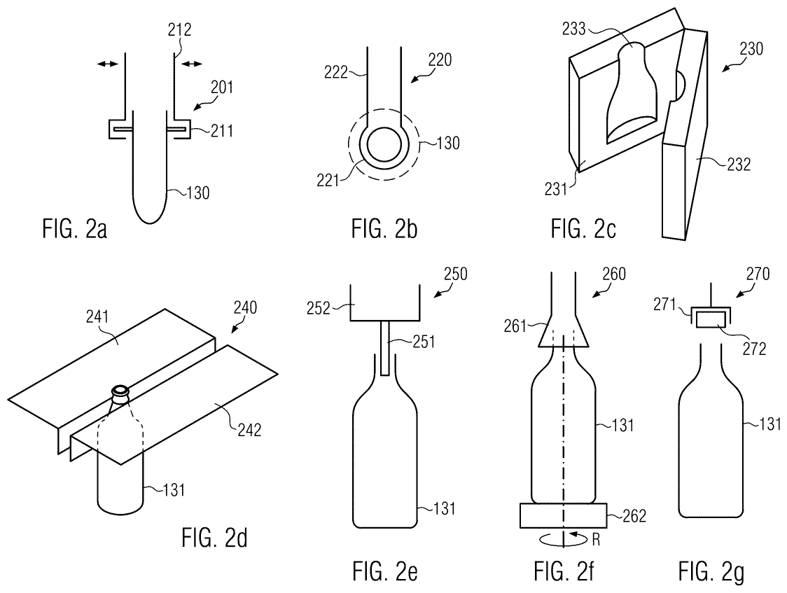

FIG. 2a-2g show various embodiments of components that may be produced using, or comprise, sterilization-proof material.

In FIG. 2a, a gripper 201 is shown, which is provided here by way of example for gripping a preform 130 on or around its support ring. Instead of the preform, the gripper 201 can also be used to hold or grip a shaped container, wherein said container can also be gripped or held at the support ring analogously to the preform.

In the embodiment shown here, the gripper comprises a gripper region 211 which comes into direct physical contact with the preform. In addition, the gripper comprises a connection region 212, which can be connected, for example, to a drive device (not shown here) in order to drive the gripper (for example to open or close it and/or to drive the gripper in a movement direction).

Preferably, at least the part of the gripper 201 that comes into contact with the preform 130 in the gripper region 211 (or the entire gripping region 211) comprises the sterilization-proof material or is made entirely therefrom. For example, it can be provided that the surface of the gripper region 211 is formed from the sterilization-proof material and an inner region of the gripper region 211 is enclosed by the surface and consists of or comprises a different material (for example plastics).

The connection region 212 may, but need not, be made of sterilization-proof material. This can, for example, save costs because the use of other materials for regions of the component that do not come into physical contact with the preform or container are less frequently sterilized and therefore do not need to be as durable.

FIG. 2b shows another embodiment in which the component comprises a clamp 220 that can clasp a preform (or container). Clamping can, for example, take place above the support ring (for example around the thread) or below the support ring or can include the support ring. Analogously to the gripper 201, the clamp 220 can comprise a clamp region 221, which comes into direct physical contact with the preform or container 130, and a connection region 222, which, for example, ensures a connection between the clamp region 221 and a drive element for actuating the clamp to release or clamp a preform. Analogously to FIG. 2a, it can also be provided here that at least the clamp region, optionally the entire clamp 220, consists of sterilization-proof material or at least comprises an outer surface made of sterilization-proof material.

In FIG. 2c, the component comprises a blow mold 230, with which containers can be produced from preforms in the blow-molding machine (see FIG. 1). The blow mold shown here is a two-part blow mold with two mold halves 231 and 232, the inner contour 233 of which determines the shape of the container to be produced. At least the inner contour 233 of the blow mold halves can comprise a surface made of sterilization-proof material that faces the preform. In order to ensure that the blow mold is stable, however, it can be provided that the blow mold halves are formed substantially in one piece, in particular consisting entirely of the sterilization-proof material, such as stainless steel. Instead of a two-part blow mold, a multi-part blow mold (for example three parts with two blow mold halves and a blow mold base part) can also be provided, the common inner contour of which defines the shape of the container to be produced. The same applies here to the provision of the sterilization-proof material as that described for the two-part blow mold.

FIG. 2d shows a further embodiment of a component 240, which comprises at least one conveying rail 241, 242. Preferably, this component 240 can be configured, for example, as an air conveyor or as part thereof, wherein a discharge device for discharging air in the transport direction (see arrow) of the containers or preforms in the air conveyor is not shown. The conveying rails may be arranged so that a container or preform can be transported therebetween, wherein the container 131 (or preform) can rest on the conveying rails 241 and 242 by its support ring. The conveying rail or rails 241 and 242 may comprise a surface facing the container made of sterilization-proof material or may be made entirely of sterilization-proof material (such as stainless steel).

In FIG. 2e, another embodiment of a component 250 in the form of a filling element is shown. The filling element 250 comprises a filling nozzle 251 which can come into direct physical contact with the container 131, although this is not necessarily the case during filling, but at least the possibility of coming into contact therewith exists. Furthermore, the filling element 250 can comprise a feed region 252 which is connected to the filling element 251, for example via lines, in order to feed the feed region 251 with the product to be poured into the container 131.

Preferably, at least the filling nozzle 251 (or a filling valve) consists of or comprises sterilization-proof material (at least its outer surface). Preferably, it can also be provided that further components of the filling element 250, in particular the feed region 252 and here in particular lines that carry product, consist of sterilization-proof material or comprise a corresponding surface (which faces the direction of the product to be guided).

FIG. 2f shows a further embodiment of a component 260. This comprises a centering device 261, which is configured here as a centering bell and can clamp the container in the region of its opening. This centering device 261 can comprise a surface made of sterilization-proof material that comes into contact with the opening region of the container 131 (for example, its mouth as far as the support ring) or can consist entirely of sterilization-proof material. In particular, it can be provided that the surface is part of a centering element that surrounds the outside of the opening region of the container or that the surface is part of a centering element that projects into the opening region of the container. In the first case, the opening region of the container is at least partially enclosed by the centering element. In the second case, the opening region at least partially surrounds the centering element and the surface is the outer surface of the centering element. The centering element may have a substantially conical shape. In the embodiment shown in FIG. 2f, the component 260 further comprises a base plate 262 on which the container 131 can be placed by its base. The base plate can also be configured as a rotary plate 262 in order to enable a container to be rotated about the axis of rotation R (shown here), optionally in conjunction with the centering device 261.

However, this embodiment is not mandatory and instead of a two-part component consisting of a centering device 261 and a base plate or rotary plate 262, only a centering device or only a base plate or rotary plate can be provided as the component 260. The base plate or rotary plate 262 may also comprise at least one outer surface made of sterilization-proof material or may consist entirely of such material. Instead of stainless steel, a sterilization-proof flexible material such as PU or plastics can be used for the surface that comes into contact with the container 131, since these have a high coefficient of friction compared with PET so that the container remains reliably held on the base plate or rotary plate and/or in the centering device.

FIG. 2g shows a component 270 in the form of a sealer. The sealer comprises a sealing element 271 that can receive a seal 272 and firmly place it on the full container 131 in order to close the full container. The sealing element 271 is preferably at least equipped with a surface made of sterilization-proof material which comes into contact with the container 131 during sealing and/or which comes into contact with the seal 272 in order to reduce any risks of contamination of the full container during sealing and/or to enable further sterilization.

Optionally, it can also be provided that a sterilization medium is applied to the seal or seals 272, which are kept in a sealer magazine (not shown in FIG. 2g), for example, in order to ensure that the sealer is sterile when applied to the container.

All of the components described can be provided individually or in combination in the plant for producing and treating containers, with any combinations of one or more of the components described also being possible.

What is claimed is:

1. A plant for producing and treating containers, the plant comprising:
a clean room;
a production machine for producing preforms;
a blow-molding machine arranged downstream of the production machine for shaping the preforms into containers; and
a container treatment machine arranged downstream of the blow-molding machine for treating the containers;
wherein the production machine, the blow-molding machine and the container treatment machine are arranged in the clean room, and wherein each component of the production machine, the blow-molding machine, and the container treatment machine that comes into physical contact with a preform or a container consists of or comprises sterilization-proof material.

2. The plant according to claim 1, wherein the sterilization-proof material is resistant to being exposed to a gaseous and/or liquid sterilization medium.

3. The plant according to claim 2, wherein the sterilization medium is H₂O₂ and/or O₃ and/or peracetic acid.

4. The plant according to claim 1, wherein the plant comprises a clean room sterilization device which can apply a sterilization medium to at least one region of the clean room.

5. The plant according to claim 1, wherein the plant comprises a preform sterilization device which can apply a sterilization medium to preforms.

6. The plant according to claim 5, wherein the plant comprises a control unit which is configured to control the preform sterilization device on the basis of a parameter relating to the application of the sterilization medium to the preforms, which parameter is indicative that the preforms are sterile.

7. The plant according to claim 6, wherein the parameter indicative that the preforms are sterile comprises a temperature at which the preforms are produced and/or a temperature at which the preforms are discharged from the production machine.

8. The plant according to claim 1, wherein the plant comprises a heating device for controlling the temperature of the preforms and wherein the heating device comprises at least one microwave radiator and/or at least one laser source and/or at least one infrared radiator.

9. The plant according to claim 1, wherein the container treatment machine comprises a filler for filling containers with a product and/or a sealer for sealing full containers and/or a labeling machine for attaching a label to a container and/or a direct printing machine for applying a printed image to a container.

10. The plant according to claim 1, wherein the plant comprises a preform cooling device for cooling preforms with a sterile first cooling medium and/or a container cooling device for cooling containers with a sterile second cooling medium.

11. The plant according to claim 1, wherein the component coming into physical contact with a preform or a container comprises at least one of a gripper, a clamp, a holding mandrel, a blow mold, a blow nozzle, a conveying rail, a filling element, a centering device, a base plate or rotary plate, a sealing element, an air conveyor arranged in the clean room for transporting containers and/or preforms.

12. A method for producing and treating containers by a plant, the plant comprising a clean room, a production machine for producing preforms, a blow-molding machine arranged downstream of the production machine for shaping the preforms into containers and a container treatment machine arranged downstream of the blow-molding machine for treating the containers, wherein the production machine, the blow-molding machine and the container treatment machine are arranged in the clean room and wherein each component of the production machine, the blow-molding machine and the container treatment machine that comes into physical contact with a preform or a container consists of or comprises sterilization-proof material;
the method comprising
producing the preforms using the production machine;
feeding the preforms to the blow-molding machine and shaping the preforms into containers; and
feeding the containers to the container treatment machine and treating the containers;
wherein the preforms and containers come into physical contact with the components.

13. The method according to claim 12, wherein the plant comprises a clean room sterilization device which can apply a sterilization medium to at least one region of the clean room, and wherein the method involves applying the sterilization medium to the region; and/or
wherein the plant comprises a preform sterilization device which can apply a sterilization medium to preforms, and wherein the method comprises applying the sterilization medium to at least one preform.

14. The method according to claim 13, wherein the plant comprises the preform sterilization device and wherein the plant comprises a control unit which is configured to control the preform sterilization device on the basis of a parameter relating to the application of the sterilization medium to the preforms, which parameter is indicative that the preforms are sterile, wherein the method involves using the control unit to control the preform sterilization device such that it applies sterilization medium to the preforms when the indicative parameter exceeds or falls below a limit value.
